(19) United States
(12) Patent Application Publication
(10) Pub. No.: US 2007/0038288 A1
(43) Pub. Date: Feb. 15, 2007

(54) Methods and apparatuses for repairing aneurysms

(76) Inventors: Whye-Kei Lye, Charlottesville, VA (US); Michael L. Reed, Charlottesville, VA (US); Mark H. Wholey, Oakmont, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/521,063

(22) PCT Filed: Jul. 11, 2003
(86) PCT No.: PCT/US03/21611

§ 371 Date: Oct. 16, 2006

Related U.S. Application Data
(60) Provisional application No. 60/395,180, filed on Jul. 11, 2002.
(60) Provisional application No. 60/421,404, filed on Oct. 24, 2002.
(60) Provisional application No. 60/421,350, filed on Oct. 24, 2002.
(60) Provisional application No. 60/428,803, filed on Nov. 25, 2002.

(51) Int. Cl. A61F 2/06; A61F 2/90

(52) U.S. Cl. 623/1.16; 623/1.36; 623/1.42

(57) ABSTRACT

Apparatuses, systems and methods are provided for repairing aneurysms in the vasculature of a patient. An aneurysm is repaired by positioning a tube or graft within the vasculature, extending through the region of the aneurysm to provide a blood flow conduit similar to the native vasculature. The tube is held in place within the vasculature by at least one expandable body having at least one microstructure. The microstructures are attached to the expandable body in a low profile fashion suitable for atraumatic introduction to the vasculature with the use of a catheter or other suitable device. Each microstructure has an end which is attached to the expandable body and a free end. Once the apparatus is positioned within the vasculature in the desired location, the one or more of the microstructures are deployed so that the free ends project radially outwardly. The free ends of the deployed microstructures then penetrate the blood vessel wall by continued expansion of the body for anchoring, reduction or migration or leakage and/or drug delivery.

### CROSS-REFERENCES TO RELATED APPLICATIONS

This application claims the benefit and priority of U.S. Provisional Patent Application No. 60/395,180 (Attorney Docket 021258-000900US) filed Jul. 11, 2002, U.S. Provisional Patent Application No. 60/421,404 (Attorney Docket 021258-000910US) filed Oct. 24, 2002, U.S. Provisional Patent Application No. 60/421,350 (Attorney Docket 021258-000700US) filed Oct. 24, 2002, and U.S. Provisional Patent Application No. 60/428,803 filed Nov. 25, 2002, the full disclosures of which are hereby incorporated by reference for all purposes.

Also, this application is related to PCT Application No. ______ (Attorney Docket 021764-000920PC), filed on the same day as this application, the full disclosure of which is hereby incorporated by reference for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK.

Not Applicable

### BACKGROUND OF THE INVENTION

The present invention relates to apparatuses, systems and methods for the treatment of aneurysms in the vasculature of patients. More particularly, the present invention relates to the treatment of abdominal aortic aneurysms.

An aneurysm is the focal abnormal dilation of a blood vessel. The complications which arise from aneurysms can include rupture, embolization, fistularisation and symptoms related to pressure on surrounding structures. Aneurysms are commonly found in the abdominal aorta, being that part of the aorta which extends from the diaphragm to the point at which the aorta bifurcates into the common iliac arteries. These abdominal aortic aneurysms typically occur between the point at which the renal arteries branch from the aorta and the bifurcation of the aorta.

When left untreated, an abdominal aortic aneurysm may eventually cause rupture of the aorta with ensuing fatal hemorrhaging in a very short time. High mortality associated with the rupture has led to the development of transabdominal surgical repair of abdominal aortic aneurysms. Surgery involving the abdominal wall, however, is a major undertaking with associated high risks. There is considerable mortality and morbidity associated with this magnitude of surgical intervention, which generally involves replacing the diseased and aneurysmal segment of blood vessel with a prosthetic device which typically includes a synthetic tube, or graft, usually fabricated of either a Dacron® polyester, a Teflon® polytetrafluoroethylene, or other suitable material.

To perform the surgical procedure, the aorta is exposed through an abdominal incision which can extend from the rib cage to the pubis. The aorta is closed both above and below the aneurysm, so that the aneurysm can then be opened and the thrombus, or blood clot, and arteriosclerotic debris removed. Small arterial branches from the back wall of the aorta are tied off The synthetic tube, or graft, of approximately the same size of the normal aorta is sutured in place, thereby replacing the aneurysm. Blood flow is then reestablished through the graft.

Disadvantages associated with the conventional surgery, in addition to the high mortality rate can include an extended recovery period associated with such surgery, difficulties in suturing the graft, or tube, to the aorta, loss of the existing aorta wall and thrombosis to support and reinforce the graft, unsuitability of the surgery for many patients having abdominal aortic aneurysms, and problems associated with performing the surgery on an emergency basis after the aneurysm has ruptured. As to the extent of recovery, a patient can expect to spend from 1 to 2 weeks in the hospital after the surgery (a major portion of which is spent in the intensive care unit) and a convalescence period at home from 2 to 3 months, particularly if the patient has other illness such as heart, lung, liver, and/or kidney disease (in which case the hospital stay is also lengthened).

A less invasive clinical approach to aneurysm repair is known as endovascular grafting. Endovascular grafting typically involves the transluminal placement of a prosthetic arterial graft within the lumen of the artery. The graft may be attached to the internal surface of an arterial wall by means of attachment devices (often similar to expandable stents), one above the aneurysm and a second below the aneurysm. Such attachment devices permit fixation of a graft to the internal surface of an arterial wall without sewing. Expansion of radially expandable stents is conventionally accomplished by dilating a balloon at the distal end of a balloon catheter. These balloon-expandable stents have found experimental and clinical application for endovascular treatments. U.S. Pat. No. 4,776,337 may be an example of such a stent. Also known are self expanding stents, such as described in U.S. Pat. No. 4,655,771 by Wallsten. These patents are hereby incorporated in their entireties, by reference.

Attachment of the device above and below the aneurysm is a conceptually straightforward procedure when the aortic aneurysm is limited to the abdominal aorta and there are significant portions of normal tissue above and below the aneurysm. Unfortunately, many aneurysms do not have suitable neck portions of normal tissue at the caudal portion (farthest from the head) of the aorta. Also, severe tortuosity of the iliac arteries and marked angulation of the aortoiliac junction can compound the difficulty of fixing the device in the caudal portion of the aorta. This situation can be exacerbated by the tendency of the abdominal aortic artery to elongate caudally during aneurysm formation. For want of sufficient normal aortic tissue to suitably attach a prosthetic graft at the caudal end of an aneurysm, or because of extension of the aneurysmal sac into the iliac arteries, bifurcated grafts have been developed that comprise a single body terminating with two limbs.

Typically, bifurcated grafts which are delivered endoluminally have an elongate flexible graft material attached to one or more anchors that support the flexible graft and serve to retain the graft in the deployed location in the blood vessel with reduced risk of the graft migrating from its deployed position. The anchor(s) is radially contractible and expandable between a reduced diameter, low profile configuration in which it can be inserted percutaneously into the patient's blood vessel and an expanded configuration in which the anchor(s) is deployed in the blood vessel and engages the inner luminal surface of the blood vessel sufficiently and in a manner to reduce the risk of the graft assembly migrating from its deployed location. In order to further reduce the risk of migration, the device may be provided with one or more hooks that can engage the wall of the blood vessel when the anchor is expanded. Although the use of such hooks is considered to be highly desirable, they may present some difficulty during delivery. For delivery, the device is contracted to a deliverable configuration. Typically the hooks extend radially outwardly which poses difficulties in both contracting the device into the deliverable configuration and in delivering the device to the blood vessel. For example, the hooks may become caught on a portion of the delivery device or may become caught with each other as the device is radially contracted. Should any of the hooks become caught, the ability of the device to properly expand upon delivery to the blood vessel may be impaired. This may interfere with the ability of the device to be positioned initially or repositioned by the delivery device. In addition, since expansion is typically achieved by release of constraining forces upon the device, the device usually self-expands as it is advanced from the confines of the delivery device. With the hooks extending radially outwardly to penetrate the vessel wall, the hooks can become caught on the delivery device or any other surface or structure as the device is self-expanding. This can damage the delivery device, the device itself and the surrounding blood vessel. Further, the addition of such hooks to the graft complicates the manufacturing process of the graft, adding additional time, cost and potential sources of failure.

It would be desirable, therefore, to provide apparatuses, systems and methods that provide the advantages of using hook-like elements to securely engage the blood vessel wall but in which the hook-like elements can be easily incorporated into the graft design, can be contracted for delivery and deployed with reduced risk of the elements becoming entangled, can provide increased resistance to graft migration and leakage, and can improve the characteristics of the surrounding tissue once in place. Further, such apparatuses and systems should not complicate the manufacturing process, reducing time, cost and potential sources of failure. It is among the general objects of the invention to provide such devices and techniques for their use.

### BRIEF SUMMARY OF THE INVENTION

The present invention provides apparatuses, systems and methods for repairing aneurysms in the vasculature of a patient. An aneurysm is repaired by positioning a tube or graft within the vasculature, extending through the region of the aneurysm to provide a blood flow conduit similar to the native vasculature. The tube is held in place within the vasculature by at least one expandable body having at least one microstructure. The microstructures are attached to the expandable body in a low profile fashion suitable for atraumatic introduction to the vasculature with the use of a catheter or other suitable device. Each microstructure has an end which is attached to the expandable body and a free end. Once the apparatus is positioned within the vasculature in the desired location, the microstructures are deployed so that the free ends project radially outwardly. The free ends of the deployed microstructures then penetrate the blood vessel wall by continued expansion of the body.

The microstructures provide a variety of functions. To begin, by penetrating the walls of the blood vessel, the microstructures firmly anchor the tube to the vessel wall therefore reducing the incidence of leaks at the time of deployment and throughout the life of the device. In addition, the microstructures reduce migration of device along the blood vessel. Such migration which could lead to leakage, exposure of the aneurysm and damage to the blood vessel, to name a few. In addition, the microstructures prevent apparent migration of the apparatus which occurs when the aneurysmal sac grows in size and as such encroaches upon the ends of the apparatus. This results in a reduction of the distance between the terminus of the apparatus and the aneurysm which is the same effect as migration. Thus, the anchoring microstructures help maintain intimate contact between the apparatus and the vessel wall and prevent aneurysmal sac growth.

The microstructures can also be used to deliver therapeutic agents to the blood vessel, the blood vessel walls and/or the outer surface of the blood vessel. Therapeutic agents such as VEGF, thrombin or collagen may be delivered into the vessel wall or deposited on the inner or outer surfaces of the vessel wall to enhance sealing by encouraging re-endothelialization and tissue regrowth or extra-cellular matrix formation. These agents may also be delivered to the aneurysmal sac. Agents such as VEGF, thrombin or collagen may also allow for tissue regrowth within the sac S, strengthening the tissue within the aneurysmal walls. Likewise, any suitable therapeutic agents may be delivered, including include drugs, DNA, genes, genes encoding for vascular endothelial growth factor, other therapeutic agents or any combination of these.

The one or more expandable bodies may be attached to the tube, such as attached to a surface of the tube wall or formed in the tube wall, or may be separate from the tube but positionable within the tube so that expansion of the expandable body penetrates the microstructures through the tube wall. In either situation, the expandable bodies may be disposed at any location along the length of the tube and may extend over a various portions of the tube, including extending along the entire tube. Likewise, microstructures may be arranged randomly or in patterns along the entire length or specific portions of the expandable bodies. For example, a plurality of microstructures may be positioned to project radially outwardly from the tube near each of its ends; this arrangement may be particularly suitable for anchoring the tube on opposite sides of the aneurysm. Other arrangements may be more suitable for other functions. For example, a plurality of microstructures may be positioned near the middle of the tube for delivery of therapeutic agents to the aneurysmal sac. Further, the deployed microstructures may project radially outwardly at various angles and to various heights. This may facilitate certain functions such as targeting specific tissue structures or layers within the vessel wall.

Although many microstructure designs are within the scope of the present invention, preferred embodiments of the microstructures have an attached end attached to the expandable body and a free end in an undeployed position, as mentioned above. In some embodiments, expansion of the body creates forces which deploy the at least one microstructure from the undeployed position to a deployed position wherein the free end projects radially outwardly. In the undeployed position, the microstructures are typically substantially aligned with an outer surface or perimeter of the body. However, it may be appreciated that the microstructures may lie beneath the surface, just so as the free ends do not project substantially outward beyond the outer surface.

In some embodiments, the at least one microstructure has a directional axis between the free end and the attached end. Each microstructure may be arranged so that its directional axis extends along the longitudinal axis, such as in a parallel manner. Alternatively, each microstructure may be arranged so that its directional axis extends across the longitudinal axis at an angle, such as in a perpendicular manner. Thus, the expansion of the body may be utilized to deploy microstructures arranged in a variety of directions, each of which generally project radially outwardly. Although the deployed microstructures may extend radially any distance from the expandable body, a distance of between 1000 μm and 5000 μm is preferred.

The free ends of the microstructures may have any desired shape. For example, in preferred embodiments the free ends have a pointed shape. When the apparatus is positioned in a blood vessel, the pointed shapes of the free ends may assist in penetration of the blood vessel wall. The shape, size and tapering of each point may possibly guide the free end to a certain penetration depth, such as to a specified tissue layer. Similarly, the free end may have an arrow-shape. This arrow shape may reduce the ability of the free end from withdrawing from a blood vessel wall once penetrated. This may be useful when the microstructures are used for anchoring. It may be appreciated that microstructures throughout the apparatus may all have the same free end shape or the shapes may vary randomly or systematically.

Exemplary embodiments of expandable bodies having deployable microstructures are described and illustrated in Provisional Patent Application No. 60/421,404 (Attorney Docket No. 021258-000910US), incorporated herein by reference for all purposes. In most embodiments, the mechanical act of expansion of the body creates forces which deploy the microstructures.

In preferred embodiments, the expandable body comprises a series of interconnected solid sections having spaces therebetween, such as resembling a conventional vascular stent. However, in contrast to conventional stents, the at least one microstructure is formed by at least one of the solid sections. Expansion of the body creates forces within the body causing mechanical deformation of the solid sections. This in turn deploys the microstructures. Since the apparatus relies upon the utilization of such mechanical deformation of the body to deploy the microstructures, additional processing beyond conventional laser machining is not necessary to create the microstructures.

In preferred embodiments, each microstructure has first and second supports and a free end, the supports affixed to associate first and second adjacent portions of the radially expandable body. Expansion of the expandable body within the patient effects relative movement between the associated first and second portions of the expandable body, the relative movement deploying the microstructures.

The expandable body can have any shape including a cylindrical shape similar to the overall shape of conventional stents. These shapes, particularly cylindrical shapes, have a circumference. Thus, relative movement of the associated first and second portions of the expandable body may comprises circumferential movement of the first portion relative to the second portion. Although the associated first and second portions may move circumferentially as the body expands, the portions may or may not be circumferentially aligned. In some embodiments wherein the associated first and second portions are in circumferential alignment, the circumferential movement of the first portion relative to the second portion draws the free end toward the circumferential alignment. In some of these and other embodiments, the circumferential movement pulls the affixed ends of the first and second supports apart which moves the free end. When the expandable body includes an interior lumen configured for receiving an expandable member, movement of the free end may create friction against the expandable member as the expandable member expands the expandable body, the friction projecting the free end radially outwardly.

In some preferred embodiments, the first and second supports comprise elongate shafts extending between the free end and the associated first and second adjacent portions of the radially expandable body. The relative movement of the associated first and second portions of the expandable body may comprise moving the associated first and second portions apart so that the supports pull the free end in opposite directions causing the free end to project radially outwardly. Often the elongate shafts are adjacent to each other and aligned with a circumference of the expandable body in the undeployed position. Thus, expansion of the body maintains the adjacent positioning of the shafts but moves them apart.

In some preferred embodiments, each microstructure further includes a third support affixed to an associated third portion of the radially expandable body, the associated first and third portions being connected so as to move in unison. Often, the first, second and third supports comprise elongate shafts attached to the free end and the associated first, second and third adjacent portions of the radially expandable body, respectively. Typically, the second support is disposed longitudinally between the first and third supports. Thus, the relative movement of the associated first and second portions of the expandable body can move the associated first and second portions apart while the associated third portion moves in unison with the associated portion so that the supports pull the free end in opposite directions forming a tripod structure which projects the free end radially outwardly.

Expansion of the expandable body may be achieved by any suitable means, such as by inflation of an expandable member, such as a balloon, within the body or by self-expansion. Typically the bodies are comprised of stainless steel, titanium, tantalum, vanadium, cobalt chromium alloys, polymers, or shape-memory alloys, such as nickel-titanium alloys, which are particularly suitable for self-expansion.

In addition, as mentioned previously, a material or therapeutic agent may be carried by the at least one microstructure, wherein the material is delivered to the patient upon deployment of the apparatus. The material may be coated on a surface of the at least one microstructure or held in a lumen within the at least one microstructure.

The systems and apparatuses of the present invention are sized for positioning within a blood vessel. Since aneurysms may be found in blood vessels of various sizes, such as ranging from small diameter cerebral arteries to large diameter regions of the aorta, embodiments of the present invention may be provided in a wide range of sizes. Likewise, the embodiments may be shaped to fit within specific anatomical geometries of the vasculature, such as bifurcations. This is particularly the case when repairing abdominal aortic aneurysms near the bifurcation of the aorta into the iliac arteries. Thus, embodiments of the present invention are provided to include legs or branches to fit within the iliac arteries or separate parts which fit within the iliac arteries and join together to form the complete apparatus in situ. Such joining may be achieved by standard methods or with the use of an additional expandable body. When an expandable body is used, the separate parts are fixed together by penetrating the microstructures through the walls of the parts. The microstructures may then optionally further penetrate the vessel wall.

Other objects and advantages of the present invention will become apparent from the detailed description to follow, together with the accompanying drawings.

### BRIEF DESCRIPTION OF THE DRAWINGS

FIG. 1 illustrates an embodiment of an apparatus of the present invention in a low profile, unexpanded state wherein the microstructures are in an undeployed position.

FIGS. 2A-2B provide cross-sectional views of the apparatus of FIG. 1 in the unexpanded and expanded states, respectively.

FIG. 3 illustrates an embodiment of the apparatus of the present invention and FIG. 3A provides an exploded view of a microstructure of FIG. 3.

FIG. 4A illustrates circumferential movement of associated first and second portions when the portions are circumferentially aligned while FIG. 4B illustrates circumferential movement of the portions when the portions are not circumferentially aligned.

FIG. 5A illustrates a representative portion of the radially expandable body having a cylindrical shape and FIGS. 5B-5C illustrate the movement of the expandable body, particularly the movement of the free ends of the microstructures as the expandable member radially expands the body.

FIGS. 6A-6C illustrate embodiments of the free ends of the microstructures of FIG. 3A.

FIGS. 6D-6G illustrate embodiments of the apparatus 10 having various designs.

FIG. 6H illustrates the embodiment depicted in FIG. 6G having the microstructures in a deployed position.

FIG. 6I provides a schematic cross sectional view of FIG. 6H.

FIG. 7 illustrates the apparatus of FIG. 1 in the expanded state wherein the microstructures are in a deployed position, extending radially outwardly from the tube.

FIG. 8 illustrates an embodiment wherein the microstructures are present near the first and second ends of the apparatus.

FIG. 9 illustrates an embodiment wherein the microstructures vary in height and in location along the length of the apparatus.

FIG. 10 illustrates an embodiment having a tube and two expandable bodies attached to the tube wall near its ends.

FIG. 11 depicts an embodiment including a tube and two removable expandable bodies which are sized for positioning within the tube.

FIG. 12 is a cross-sectional view of the embodiment of FIG. 7 illustrating the penetration of the microstructures through the tube and into the surrounding vessel wall.

FIG. 13 illustrates an aneurysm within a blood vessel and an apparatus of the present invention positioned across the aneurysmal sac.

FIG. 14 illustrates an apparatus of the present invention positioned across the aneurysmal sac and the delivery of therapeutic agents to this sac through microstructures.

FIGS. 15-16 illustrate embodiments of the present invention shaped to fit with an abdominal aorta traversing a bifurcation.

FIG. 17 illustrates the embodiment of FIG. 16 positioned within an abdominal aortic aneurysm.

FIGS. 18A-18C illustrate an expandable body used to provide structural support to the apparatus and reduction of leakage around the apparatus.

FIGS. 19-20 illustrate embodiments of the present invention including extension cuffs.

### DETAILED DESCRIPTION OF THE INVENTION

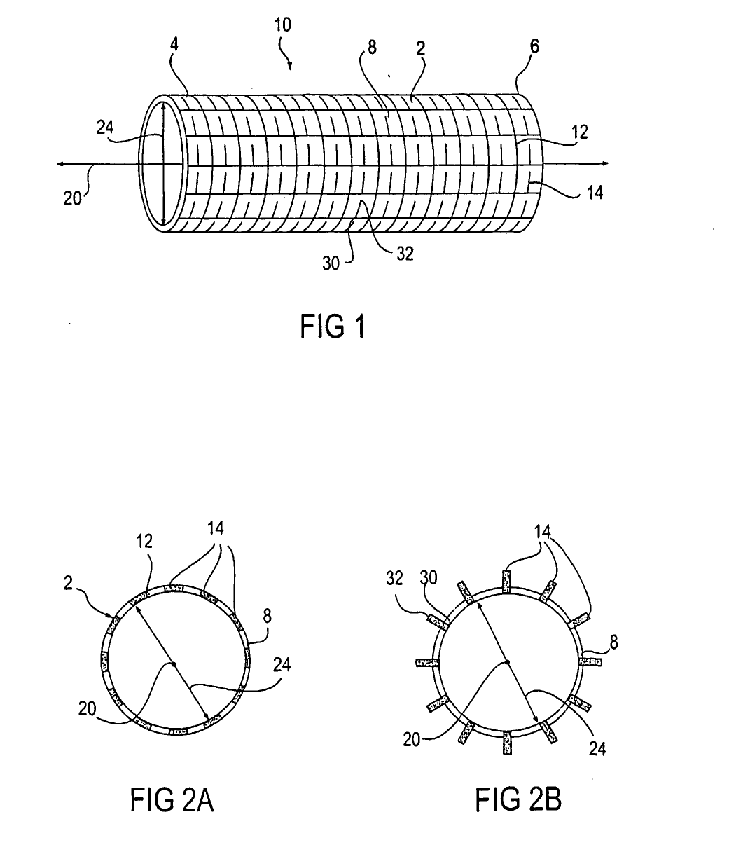

The following detailed description illustrates the invention by way of example, not by way of limitation of the principles of the invention. Referring to FIG. 1, an embodiment of an apparatus 10 of the present invention is illustrated, the apparatus 10 comprises a tube 2 having a first end 4, a second end 6 and a tube wall 8 extending between the first and second ends 4, 6. In addition, the apparatus 10 comprises an expandable body 12 attached to the tube wall 8 including at least one microstructure 14. Each microstructure 14 has an attached end 30 attached to the body and a free end 32 in an undeployed position. FIG. 1 illustrates the apparatus 10 in an unexpanded state wherein the microstructures 14 are in an undeployed position. Here, the microstructures 14 are preferably aligned or flush with an outer surface of the apparatus 10 so that the surface does not include substantial protrusions. Alternatively, the microstructures 14 may be positioned below the surface of the apparatus 10. FIG. 1 also shows cross-sectional diameter 24 and longitudinal axis 20.

The tube 2 preferably has a generally, circular cross-sectional configuration, and may be made from a variety of materials. Examples of such materials are Dacron® and other polyester materials, Teflon® (polytetrafluoroethylene), Teflon® coated Dacron® material and porous polyurethane, to name a few. Generally, the tube material possesses the requisite strength characteristics to be utilized as a vascular graft, particularly an aortic graft when used to repair abdominal aortic aneurysms, as well as have the requisite compatibility with the human body in order to be used as a graft, or implant material, without being rejected by the patient's body. The material can be knitted or woven, and can be warp or weft knitted. If the material is warp knitted, it may be provided with a velour, or towel like surface, which may speed up clotting of blood upon contact with the tube 2 in order to increase the attachment, or integration, of tube 2 to the vessel or aorta. When the apparatus 10 is utilized to repair an aneurysm, such as to create an artificial conduit, it would be preferable to make tube 2 of a fluid impervious material.

The expandable body 12 typically comprises a series of interconnected solid sections having spaces therebetween. The solid sections are comprised of stainless steel, shape memory alloys, titanium, tantalum, vanadium, cobalt chromium alloys, polymers, or a combination of these. Thus, the expandable body 12 forms a type of scaffolding which is attached to the tube 2. The body 12 may be attached to the outside of the tube 2, the inside of the tube 2, and/or attached in a manner so that it lies within the wall 8 of the tube 2.

FIGS. 2A-2B provide cross-sectional views of the apparatus 10 of FIG. 1 in the unexpanded and expanded states, respectively. FIG. 2A shows the tube wall 8 and the attached expandable body 12 having microstructures 14, highlighted by shading. Thus, when the expandable body 12 is in the unexpanded state, the microstructures 14 are in an undeployed position as shown. FIG. 2B illustrates the expandable body 12 in an expanded state wherein the cross-sectional diameter 24 is increased. Here, the microstructures 14 are in a deployed position wherein a free end 32 of each microstructure 14 projects radially outwardly from the tube 2 while an attached end 30 remains attached to the body 12. It may be appreciated that the deployed microstructures 14 may form any angle with the surface of the tube 2, including a substantially 90 degree angle as shown. Further, different microstructures 14 may form different angles, angles may vary randomly or in a pattern, angles may be selectable particularly based on amount of expansion, and some microstructures may not deploy while others deploy.

The expandable body 12 of the present invention may resemble conventional 30 stents and may be similarly manufactured, however the particular design of the structure is dependent, in part, upon the microstructures and the way that they deploy upon expansion of the body 12. As mentioned previously, exemplary embodiments of expandable bodies having deployable microstructures are described and illustrated in Provisional Patent Application No. 60/421,404 (Attorney Docket No. 021258-000910), incorporated herein by reference for all purposes. In most embodiments, the mechanical act of expansion of the body 12 creates forces which deploy the microstructures 14.

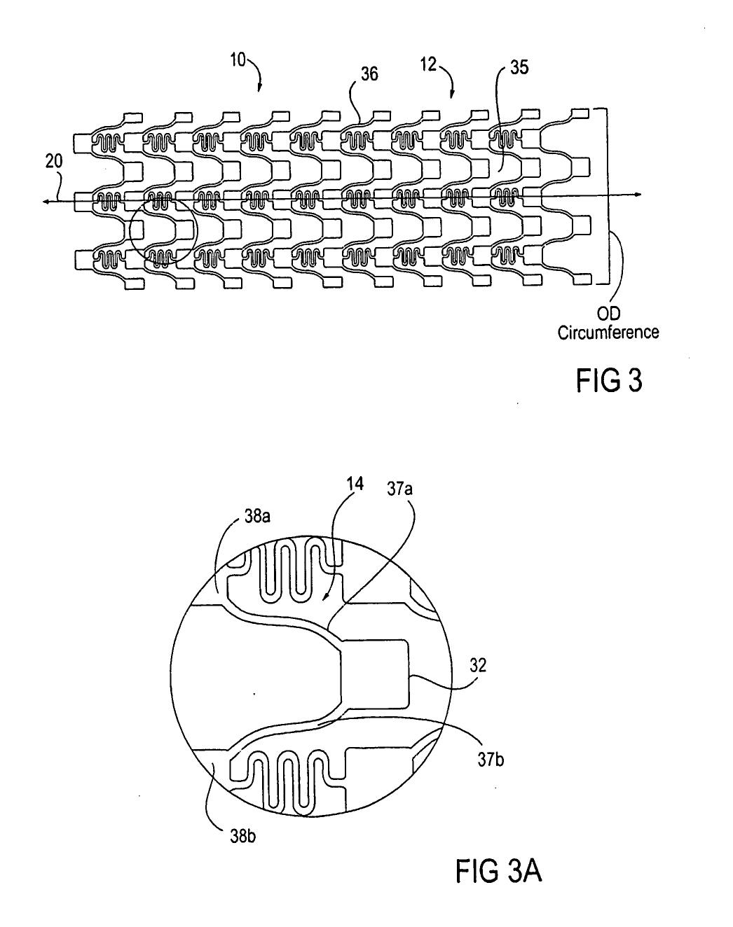

FIG. 3 illustrates an embodiment of the apparatus 10. Although the apparatus 10 is illustrated in a flat plane, it is formed cylindrically around longitudinal axis 20 in this embodiment. As shown, the expandable body 12 comprises a series of interconnected solid sections 36 having spaces 35 therebetween. A portion of the body 12 including a microstructure 14 is illustrated in exploded view in FIG. 3A. Each microstructure has a first support 37a, a second support 37b and a free end 32. The supports 37a, 37b are affixed to associate first and second adjacent portions 38a, 38b of the radially expandable body.

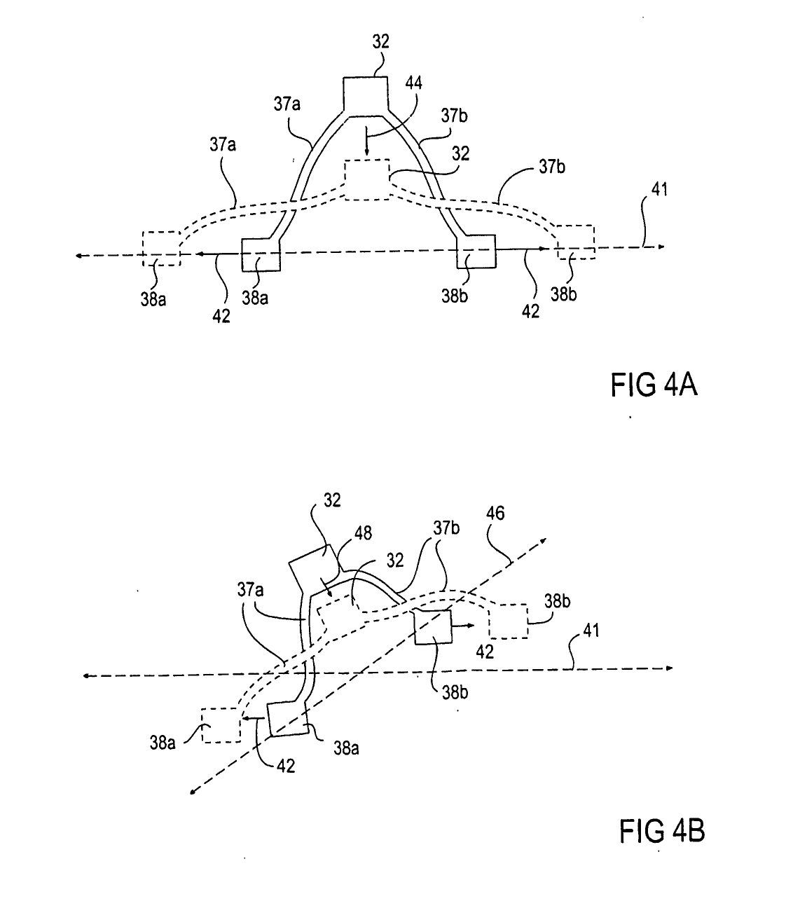

Referring to FIG. 4A, the associated first and second portions 38a, 38b may be in circumferential alignment, as illustrated by dashed line 41. It may be appreciated that dashed line 41 wraps around to form a circular shape when following the circumference of a cylindrical body, however the dashed line 41 is illustrated as a straight line for clarity. When the expandable body expands radially, the relative movement of the associated first and second portions 38a, 38b may comprise circumferential movement of the first portion 38a relative to the second portion 38b, as indicated by arrows 42. When the associated first and second portions 38a, 38b may be in circumferential alignment, as shown, the circumferential movement of the first portion 38a relative to the second portion 38b draws the free end 32 toward the circumferential alignment or line 41, as indicated by arrow 44.

Referring to FIG. 4B, the associated first and second portions 38a, 38b may be in noncircumferential aligmnent, as illustrated by dashed line 46 which forms an angle with line 41 representing circumferential alignment. Thus, when the expandable body expands radially, the relative movement of the associated first and second portions 38a, 38b may still comprise circumferential movement of the first portion 38a relative to the second portion 38b, as indicated by arrows 42. And, the circumferential movement of the first portion 38a relative to the second portion 38b pulls the affixed ends of the first and second supports 37a, 37b apart which moves the free end 32. However, in this situation, the free end is no longer drawn toward the circumferential alignment, rather the free end is drawn toward line 46 as indicated by arrow 48.

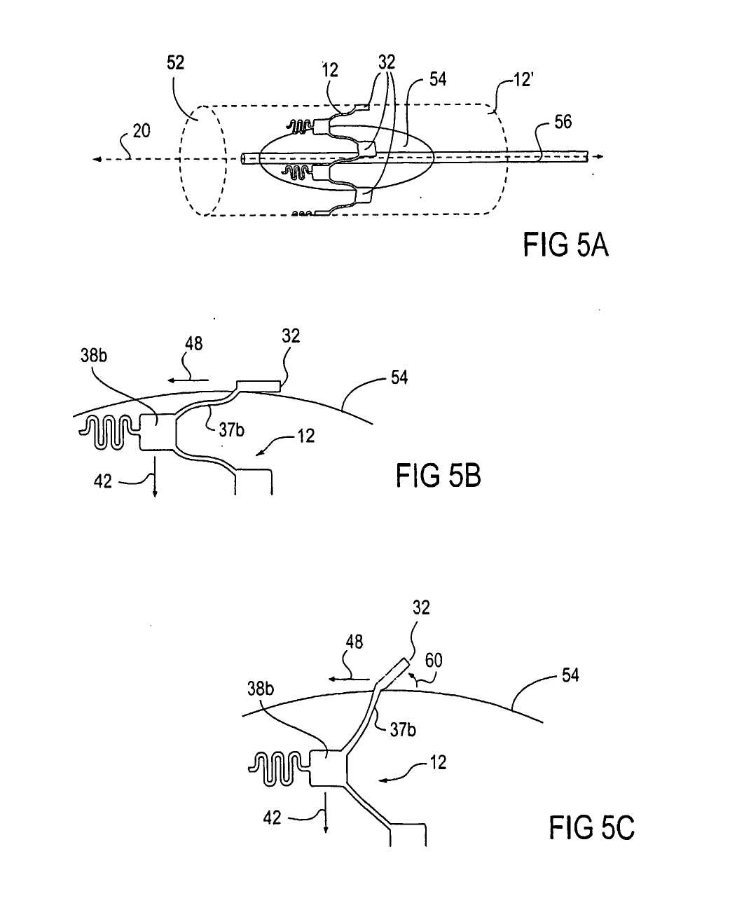

FIG. 5A illustrates a representative portion of the radially expandable body 12 having a cylindrical shape, the remainder of the body illustrated by dashed body 12′. In this embodiment the radially expandable body 12 further comprises an interior lumen 52 along the longitudinal axis 20. The interior lumen 52 is configured for receiving an expandable member 54 which expands the expandable body 12. Typically, the expandable member 54 is mounted on a catheter 56. FIGS. 5B-5C illustrate the movement of the expandable body, particularly the movement of the free ends 32 of the microstructures 14 as the expandable member 54 radially expands the body 12. FIG. 5B is a side view of a portion of the expandable body 12, including a microstructure 14, mounted on expandable member 54. Expansion of the expandable member 54 effects relative movement between the associated first and second portions 38a, 38b, in this case such expansion effects circumferential movement. Circumferential movement is indicated by arrow 42. It may be appreciated that the associated first portion 38a is not shown in FIG. 5B since FIG. 5B is a side view and portion 38a would be located symmetrically on the backside of the expandable member 54. The circumferential movement pulls the affixed ends of the first and second supports 37a, 37b apart which moves the free end 32, indicated by arrow 48. As shown in FIG. 5C, such movement of the free end 32 projects the free end 32 radially outwardly, as indicated by arrow 60. Such projection may be due to friction created between the free end 32 and the expandable member 54 as the expandable member 54 expands the expandable body 12. Alternatively, such projection may be due to other factors, such as the direction of movement of the supports 37a, 37b, the shape of the supports 37a, 37b, or a combination of factors.

It may be appreciated that the expandable body 12 of FIGS. 5A-5C may alternatively be expanded by means other than expansion by an expandable member 54. For example, the expandable body 12 may be self-expanding, as previously mentioned. In this situation, the expandable body 12 is pre-formed so that deployment of the body 12 allows the body 12 to self-expand toward a predetermined configuration. Pre-forming may be achieved with the use of an expandable member 54, wherein the body 12 is set while surrounding an expandable member 54 so as to later form this configuration. When the expandable body 12 expands within the body, projection of the microstructures may be due torqueing or movement of the supports 37a, 37b, for example.

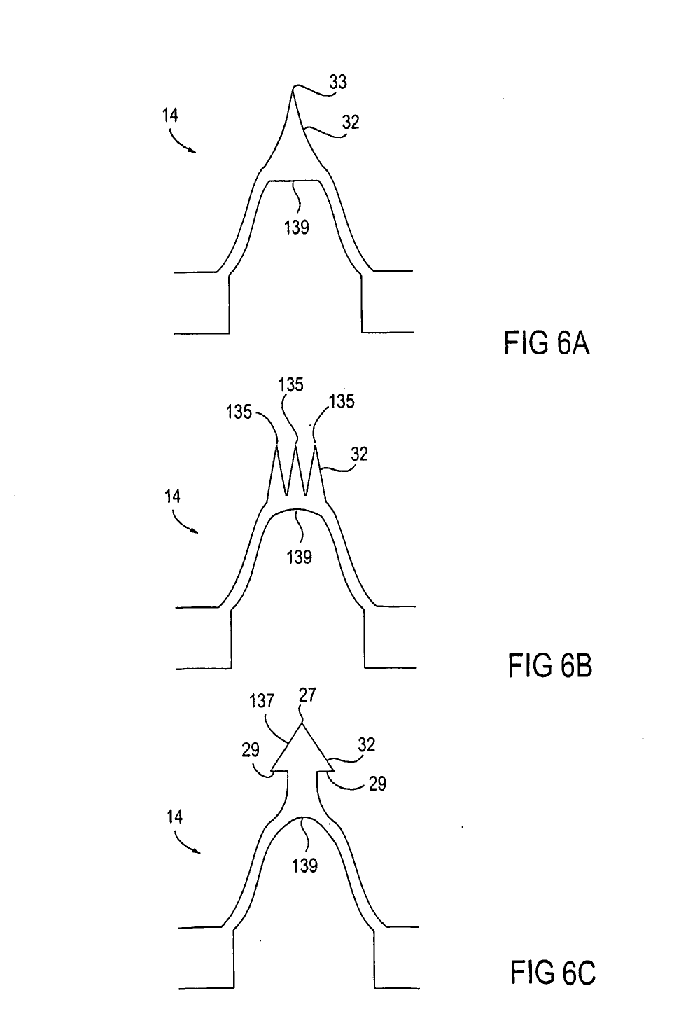

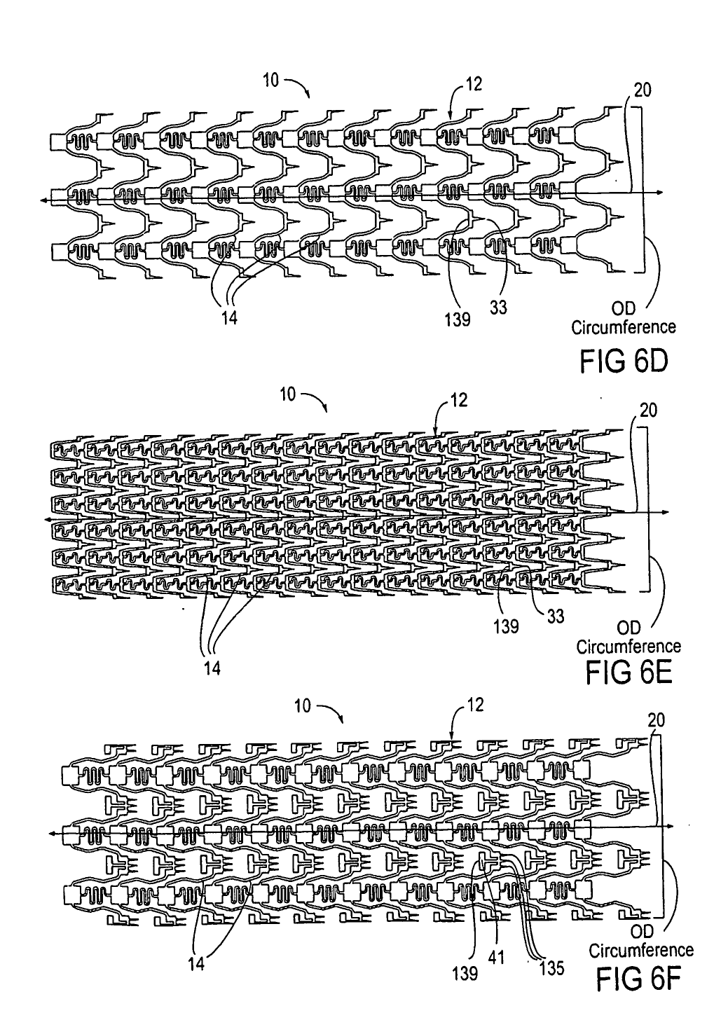

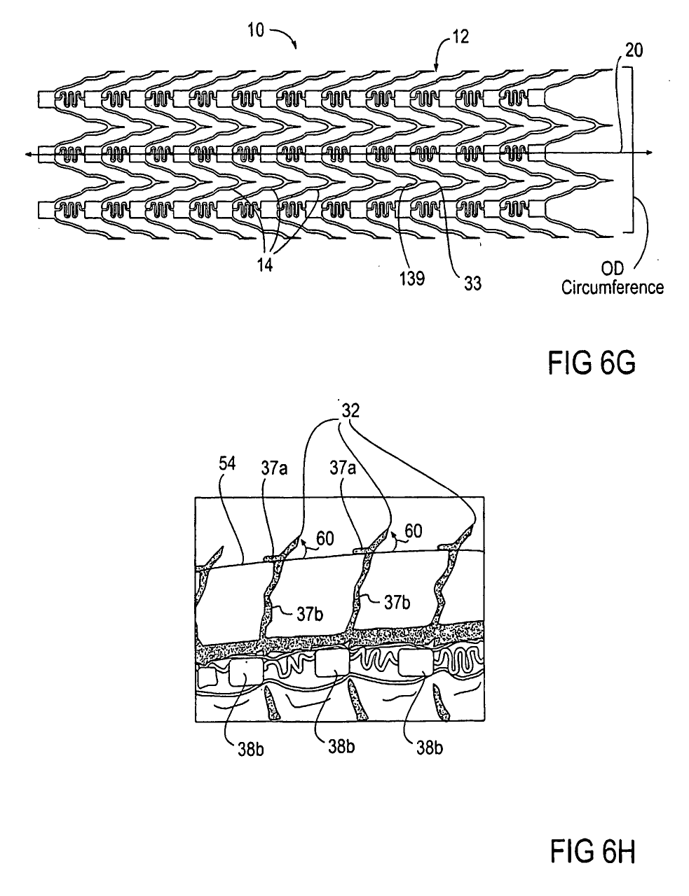

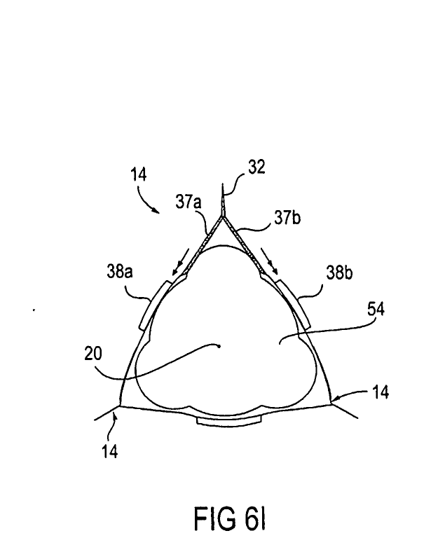

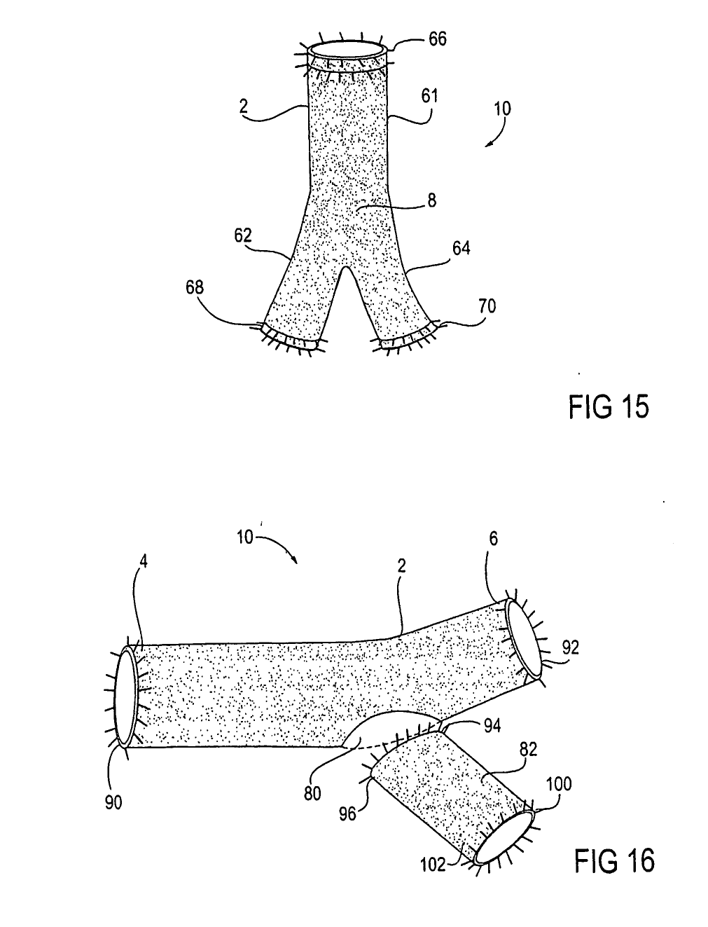

The free ends 32 of the microstructures 14 depicted in FIGS. 3, 3A, 4A-4B, 5A-5C are each shown to have a flat-edged shape. However, the free ends 32 may have any desired shape. For example, FIGS. 6A-6C illustrate additional embodiments of microstructures 14 having different shaped free ends 32. In each of these embodiments, the free ends 32 have a pointed shape. When the apparatus 10 is positioned in a body lumen, such as a blood vessel, the pointed shapes of the free ends 32 may assist in penetration of the lumen wall. The shape, size and tapering of each point may possibly guide the free end 32 to a certain penetration depth, such as to a specified tissue layer. In FIG. 15A, the free end 32 has a single point 33 and in FIG. 6B the free end 32 has multiple points 135. In FIG. 6C, the free end 32 has an arrow-shaped point 137. The arrow-shaped point 137 includes a pointed tip 27 and at least one undercut 29 to reduce the ability of the free end 32 from withdrawing from a lumen wall once penetrated. This may be useful when the microstructures are used for anchoring. It may be appreciated that microstructures 14 throughout the apparatus 10 may all have the same free end 32 shape or the shapes may vary randomly or systematically. Likewise, the free end 32 may have a flat-shaped inner edge 139, as illustrated in FIG. 6A, to maximize friction against an expandable member 54 or the free end 32 may have various other shaped inner edges 139, as illustrated in FIGS. 6B-6C.

FIGS. 6D-6F illustrate embodiments of the apparatus 10 having various designs. Again, although the apparatus 10 is illustrated in a flat plane, it is formed cylindrically around longitudinal axis 20 in each embodiment. In FIG. 6D, the microstructures 14 have free ends 32 which are shaped as a single point 33 and include a flat inner edge 139. Thus, the free ends 32 are similar to the embodiment illustrated in FIG. 6A. FIG. 6E also illustrates an embodiment wherein the microstructures 14 have free ends 32 which are shaped as a single point 33 and include a flat inner edge 139. However, in this embodiment, the microstructures 14 are positioned more closely together, in a denser pattern. In FIG. 6F the microstructures 14 have free ends 32 which are shaped to have multiple points 135 and to include a flat inner edge 139. In addition, the flat inner edge 139 is part of a flange 41 which is directed opposite of the points 135. The flange 43 provides a wide flat inner edge 139 to maximize friction against an expandable member 54 and a narrow neck region 45 to enhance flexibility and rotation of the multiple points 135 radially outwardly.

FIG. 6G illustrates an embodiment of the expandable body 12 wherein the free ends 32 of the microstructures 14 have a single point 33 and curved inner edge 139. And, FIG. 6H illustrates the microstructures of FIG. 6G in a deployed position. FIG. 6H provides a view similar to FIG. 5C wherein circumferential movement pulls the affixed ends of the first and second supports 37a, 37b apart which moves the free end 32. Such movement of the free end 32 projects the free end 32 radially outwardly, as indicated by arrow 60. As mentioned, such projection may be due to friction created between the free end 32 and the expandable member 54 as the expandable member 54 expands the expandable body 12.

Alternatively, such projection may be due to other factors, such as the direction of movement of the supports 37a, 37b, the shape of the supports 37a, 37b, or a combination of factors. For example, FIG. 6I provides a schematic cross sectional view of FIG. 6H. Prior to expansion, the free ends 32 and associated first and second portions 38a, 38b of the expandable member 12 lie substantially equidistant from the longitudinal axis 20. Upon expansion of the expandable member 54, the forces are applied to the first support 37a and second support 37b. Upon further inflation, the first and second supports 37a, 37b present less resistance to the expandable member 54, and as such the expandable member 54 expands more in the regions spanned by the first and second supports 37a, 37b than in the regions of the associated first and second portions 38a, 38b, as illustrated in FIG. 6I. This deploys the microstructures 14 since there is a contact point between the first and second supports 37a, 37b and the expandable member that serves as a fulcrum about which moment is generated as the expandable member continues to expand. The resulting moment further projects the microstructure radially outwardly.

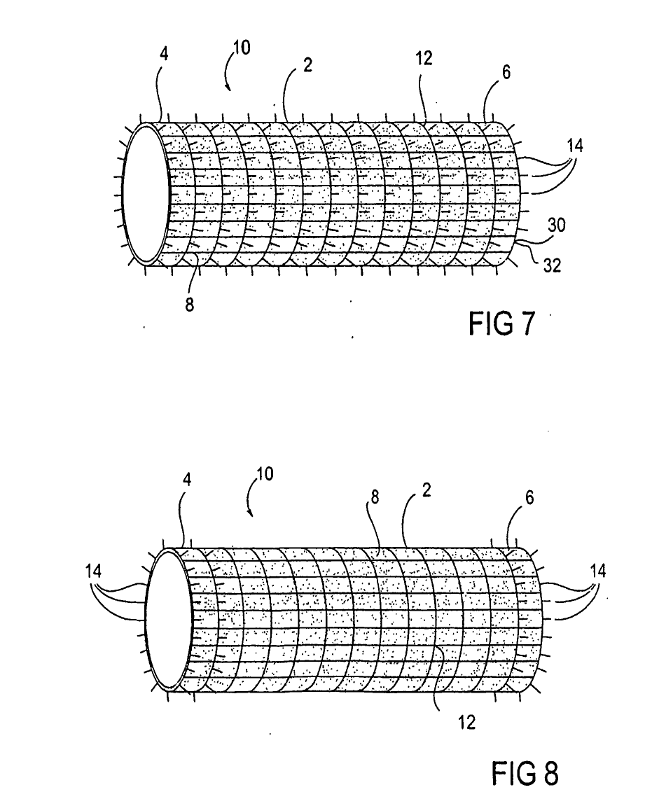

It may be appreciated that any number of microstructures 14 may be present and may be arranged in a variety of patterns along the entire length of the body 12 or along any subportion. For example, FIG. 7 illustrates the embodiment of FIG. 1 wherein the microstructures 14 are present along the entire length of the body 12 and the body 12 extends along the entire length of the tube 2. In addition, FIG. 7 illustrates the microstructures 14 in the deployed position wherein the free ends 32 project radially outwardly from the tube 2. Alternatively, the microstructures 14 may be present in select locations, such as near the first end 4, near the second end 6, or near both ends 4, 6 as illustrated in FIG. 8, while the body 12 extends along the entire length of the tube. These particular arrangements of microstructures 14 may be useful in anchoring the apparatus 10 across an aneurysm, as will be described in more detail in later sections.

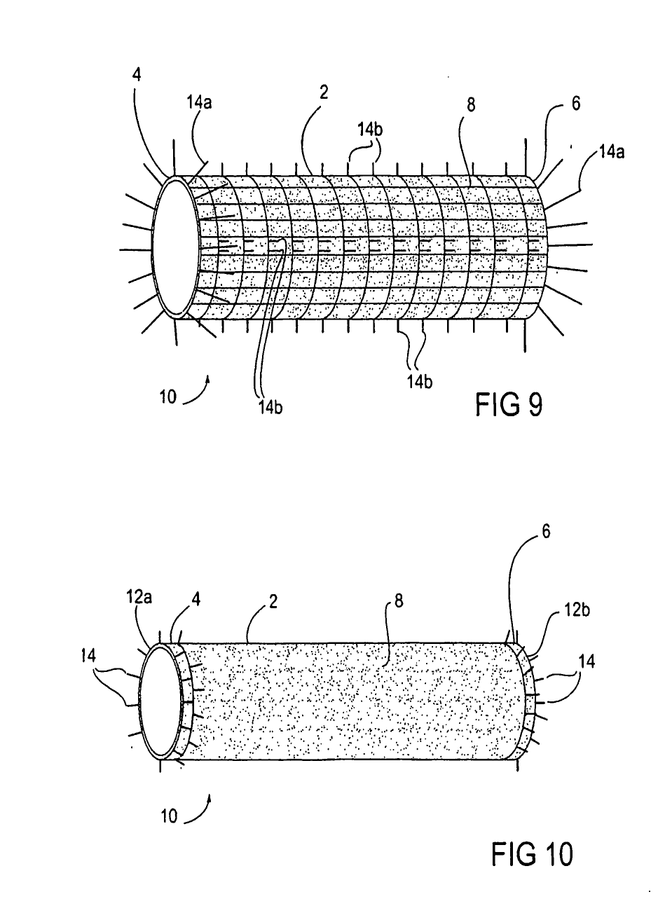

In addition, the deployed microstructures 14 may vary in height and in location. FIG. 9 illustrates an embodiment having longer microstructures 14a located near the first end 4 and second end 6 which extend further from the tube 2 in the deployed position than shorter microstructures 14b located between the ends 4, 6. This may be useful in a variety of treatment situations. For example, the longer microstructures 14a may be sized to traverse a thickness of compressed plaque, penetrate the vascular lumen to a desired depth, or to pass completely through the lumen wall. The location of these longer microstructures 14a may be helpful in anchoring the apparatus 10 within the vascular lumen and/or delivering therapeutic agents into or around the lumen walls. In this embodiment, the shorter microstructures 14b are located in strips between the first end 4 and second end 6. The location and size of the shorter microstructures 14b may be useful to deliver therapeutic agents to the vascular lumen or to an aneurysmal sac.

Referring now to FIG. 10, an embodiment of the apparatus 10 of the present invention is illustrated which includes a tube 2 and two expandable bodies 12a, 12b attached to the tube wall 8, wherein each expandable body includes at least one microstructure 14 as described above. Here, one expandable body 12a is attached near the first end 4 of the tube 2 and the other expandable body 12b is attached near the second end 6. Thus, the tube wall 8 extends between the bodies 12a, 12b. Thus, this embodiment is similar to that depicted in FIG. 8, however in this embodiment the expandable bodies 12a, 12b do not extend the length of the tube 2. This may be desirable in certain treatment situations.

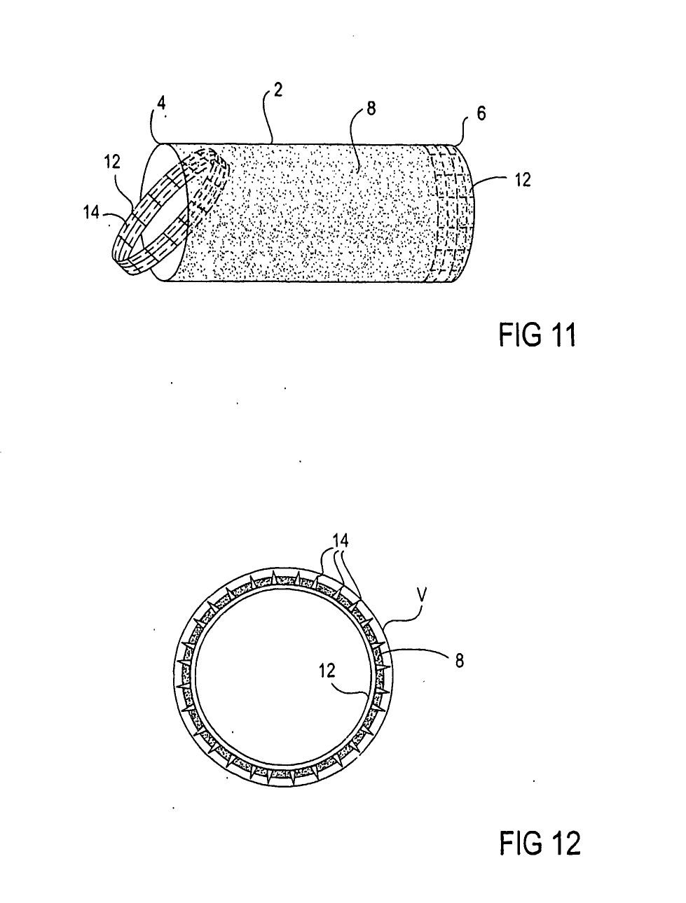

Referring now to FIG. 11, an embodiment of a system of the present invention is provided including a tube 2 having a first end 4, a second end 6 and a tube wall 8 extending between the first and second ends 4, 6, and at least one expandable body 12 which is sized for positioning within the tube 2. Here, two expandable bodies 12a, 12b are shown, a first expandable body 12a partially in place near the first end 4 to illustrate its moveability and a second expandable body 12b in place near the second end 6. Thus, the at least one expandable body 12 may be positioned at any location along the length of the tube 2, including extending beyond the ends 4,6 of the tube. The at least one expandable body 12 may also be removed and repositioned due to its moveability.

When the expandable body 12 is positioned within the tube 2, expansion of the body 12 and deployment of the microstructures 14 occurs within the tube 2 so that further expansion penetrates the microstructures 14 through the tube wall 8, as illustrated in FIG. 12. FIG. 12 provides a cross-sectional view of the expandable body 12 within a tube 2 and illustrates a plurality of microstructures 14 penetrating the tube wall 8. FIG. 12 also illustrates the microstructures 14 further penetrating a surrounding blood vessel wall V. Thus, the microstructures 14 may be used to anchor the apparatus 10 within the blood vessel or to deliver therapeutic agents to the blood vessel in a manner similar to that in which the expandable body 12 is attached to the outside surface of the tube 2.

As mentioned previously, the present invention may be utilized for any sort of treatment which involves delivery of a therapeutic agent and/or anchoring of a device. The devices could be introduced into various body lumens, such as the vascular system, lungs, gastrointestinal tract, urethra or ureter. The function of the microstructures includes but is not limited to facilitating drug and gene delivery, securing the device in place and providing a mechanical seal to the lumen wall. Thus, the present invention is particularly suited for repair of aneurysms within the vascular system.

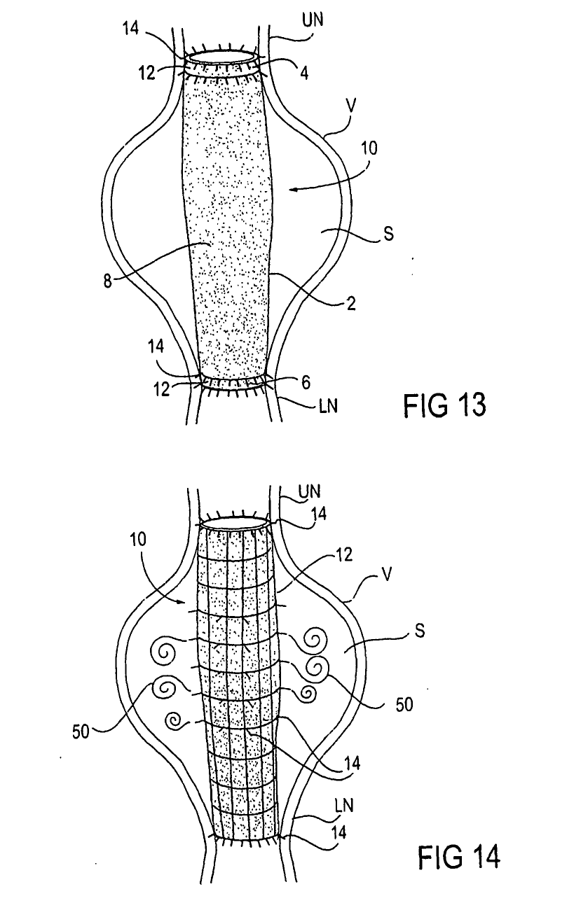

FIG. 13 illustrates an aneurysm within a blood vessel V. An aneurysm comprises a sac S caused by abnormal dilation of the wall of the blood vessel V and may occur within any blood vessel in the body. Life-threatening aneurysms can occur in cerebral blood vessels and the aorta, to name a few. Repair of such aneurysms typically involves bridging the sac S with a graft material, wherein the graft is at least secured to the upper neck UN and lower neck LN of the blood vessel V near the ends of the sac S. This provides a conduit for blood flow through the blood vessel V, preventing further collection of blood in the aneurysmal sac S and reducing the progression of growth of the aneurysm and the risk of sac rupture due to blood pressure.

Positioning of the apparatus of the present invention is typically performed via standard catheterization techniques. These methods are well known to cardiac physicians and are described in detail in many standard references. In brief, percutaneous access of the vasculature is obtained with standard needles, guide wires, sheaths, and catheters. After engagement of the blood vessel containing the aneurysm with a hollow guiding catheter, a guidewire is passed across the portion of the blood vessel where the apparatus is to be deployed. The apparatus is then passed over this guidewire, using standard coronary interventional techniques, to the site where the apparatus is to be deployed. Typically, this site is within the aneurysm so that the apparatus 10 straddles the aneurysm, extending between the upper neck UN and the lower neck LN as illustrated in FIG. 13. The expandable body 12 or bodies are expanded, deploying the microstructures 14 and forcing the microstructures 14 through the wall of the blood vessel V to anchor the apparatus in place. In the embodiment of FIG. 13, expandable bodies 12 are located near the first end 4 and second end 6 of the tube 2 so that the microstructures 14 penetrate the upper neck UN and lower neck LN on opposite sides of the aneurysmal sac S.

The microstructures 14 improve the performance of the apparatus 10 in a variety of ways. For instance, the microstructures 14 firmly anchor the apparatus to the vessel wall therefore reducing the incidence of leaks at the time of deployment of the apparatus. Also, the pressure from the blood flow through the apparatus further reduces migration and prevents leakage from the apparatus over time. In addition, the microstructures prevent apparent migration which occurs when the aneurysmal sac grows in size and as such encroaches upon the first and second ends of the apparatus. This results in a reduction of the distance between the terminus of the apparatus and the aneurysm. Thus, the anchoring microstructures help maintain intimate contact between the apparatus and the vessel wall and prevent aneurysmal sac growth.

The microstructures 14 can also be used to delivery therapeutic agents. Therapeutic agents such as VEGF, thrombin or collagen may be delivered into the vessel wall or deposited on the inner or outer surfaces of the vessel wall to enhance sealing by encouraging re-endothelialization and tissue regrowth or extra-cellular matrix formation. These agents may also be delivered to the aneurysmal sac S, as illustrated in FIG. 14. In this embodiment, the expandable body 12 extends the length of the tube wall 8 and has microstructures 14 near the first end 4 and second end 6 to anchor the apparatus 10 in place and has microstructures 14 between the ends 4, 6 for delivery of therapeutic agents 50 to the aneurysmal sac S. Agents such as VEGF, thrombin or collagen may also allow for tissue regrowth within the sac S, strengthening the tissue within the aneurysmal walls. It may be appreciated that any suitable therapeutic agents may be delivered, including include drugs, DNA, genes, genes encoding for vascular endothelial growth factor, other therapeutic agents or a combination of these to be delivered to the lumen wall for therapeutic purposes.

The present invention may be particularly suitable for repair of abdominal aortic aneurysms. An abdominal aortic aneurysm is a sac caused by an abnormal dilation of the wall of the aorta, a major artery of the body, as it passes through the abdomen. The abdomen is that portion of the body which lies between the thorax and the pelvis. It contains a cavity, known as the abdominal cavity, separated by the diaphragm from the thoracic cavity and lined with a serous membrane, the peritoneum. The aorta is the main trunk, or artery, from which the systemic arterial system proceeds. It arises from the left ventricle of the heart, passes upward, bends over and passes down through the thorax and through the abdomen to about the level of the fourth lumbar vertebra, where it divides into the two common iliac arteries at a bifurcation.

To treat abdominal aortic aneurysms, the apparatus 10 is shaped to be disposed at least partially within the aneurysm. In particular, the tube 2 is shaped to fit the aortic geometry. For example, FIG. 15 illustrates an embodiment of the apparatus 10 of the present invention shaped to fit within the abdominal aorta, traversing the bifurcation. Thus, the tube 2 includes a main shaft 61, a first leg 62, and a second leg 64. This embodiment further includes three expandable bodies, a first expandable body 66 disposed near the end of the main shaft 61, a second expandable body 68 disposed near the end of the first leg 62 and a third expandable body 70 disposed near the end of the second leg 64, as shown. Positioning of these expandable bodies 66, 68, 70 are intended to provide anchoring for the apparatus within the aorta and iliac arteries surrounding the abdominal aortic aneurysm. Alternatively, one or more expandable bodies may extend over larger portions of the tube wall 8, including over the entire tube 2.

In another example, shown in FIG. 16, the apparatus 10 of the present invention is also shaped to fit within the abdominal aorta, traversing the bifurcation. In this example, the apparatus 10 comprises a tube 2 having a first end 4 and a second end 6 and further includes an opening 80 between the first and second ends 4, 6 to align with one of the iliac arteries. An additional tube 82, shaped to be disposed within the one of the iliac arteries, may then be inserted into the opening 80. This embodiment includes four expandable bodies, a first expandable body 90 disposed near the first end 4 of the tube 2, a second expandable body 92 disposed near the second end 6 of the tube 2, a third expandable body 94 disposed near a first end 96 of additional tube 82 and a fourth expandable body 100 disposed near a second end 102 of additional tube 82. Here, the first, second and fourth expandable bodies 90, 92, 100 are intended to provide anchoring for the apparatus within the aorta and iliac arteries surrounding the abdominal aortic aneurysm. The third expandable body 94 is intended to attach the additional tube 82 to the tube 2 in the area of the opening 80. Thus, expansion of the body 94 deploys the microstructures 14 and penetrates the microstructures 14 into the wall 8 of the tube. The microstructures 14 may be sized and/or oriented so as to penetrate the wall 8 without passing entirely through the wall 8. Alternatively, the wall 8 may be constructed of one or more materials or in a manner to prevent complete passage of the microstructures through the wall 8. However, in some embodiments it may be desired that the microstructures 14 pass through the wall 8 and further penetrate the aortic wall to provide further anchoring and/or delivery of agents.

An expandable body may alternatively be positioned around the opening 80 on or within the tube 2 to attach the tube 2 to the additional tube 82. And, in general, it may be appreciated that any number of expandable bodies may be used. In particular, the additional tube 82 may be joined to the tube 2 without the use of the third expandable body 94.

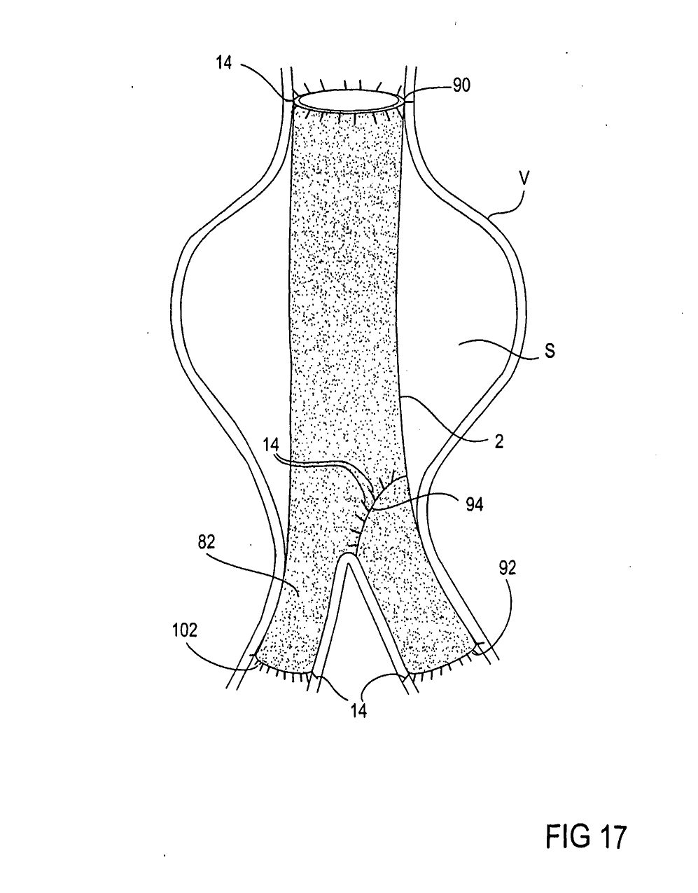

FIG. 17 illustrates the embodiment of FIG. 16 positioned within an abdominal aortic aneurysm. Here, the tube 2 extends from the upper neck UN to one of the iliac arteries IA. The tube 2 has an opening 80 aligned with the other iliac artery IA′. An additional tube 82 is positioned within the other iliac artery IA′ and extended through opening 80. The third expandable body 94 is shown wherein the microstructures 14 are deployed to attach the tube 2 to the additional tube 82 near the opening 80. Likewise, the first, second and fourth expandable bodies 90, 92, 100 are expanded so that the deployed microstructures 14 penetrate the walls of the blood vessel V and provide anchoring for the apparatus 10 within the aorta and iliac arteries surrounding the abdominal aortic aneurysmal sac S. Again, the microstructures 14 may also provide delivery of agents to the blood vessel V, areas within or surrounding the blood vessel, and within the aneurysmal sac S.

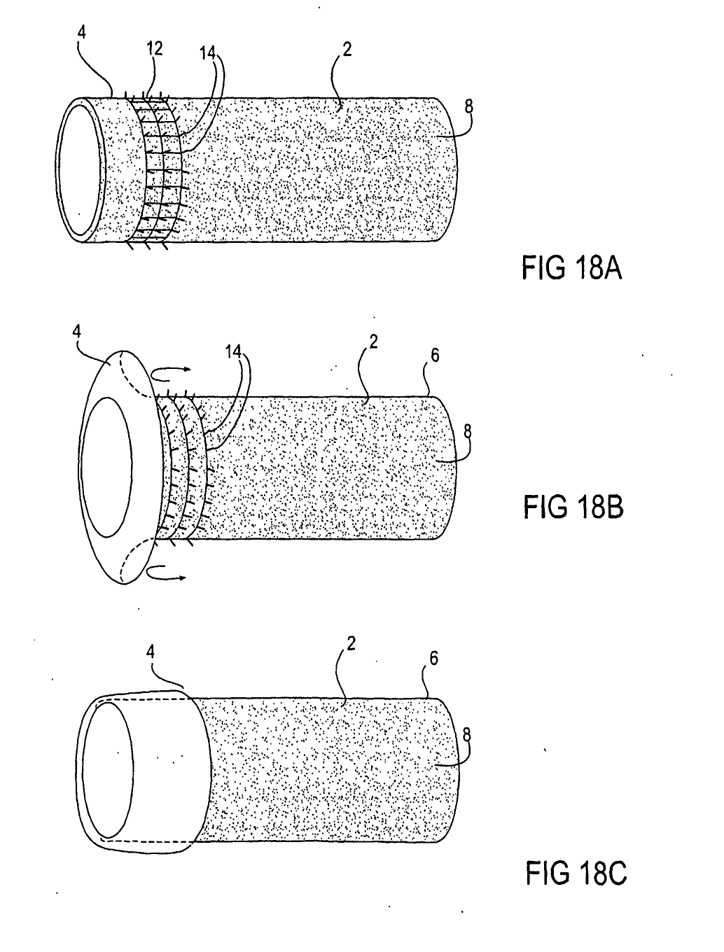

Referring now to FIGS. 18A-18C, an expandable body may be used to provide structural support to the apparatus and reduction of leakage around the apparatus. FIG. 18A illustrates a tube 2 with an expandable body 12 mounted externally near its first end 4. Here, the microstructures 14 are deployed to extend radially outwardly from the tube 2. As illustrated in FIG. 18B, the first end 4 of the tube 2 may then be inverted and folded back toward the second end 6 of the tube 2. As shown in FIG. 18C, the inverted first end 4 may cover the expandable body 12 so that microstructures 14 penetrate or pierce the wall 8 of the first end 4 of the tube 2 holding the first end 4 in place in the inverted position. When positioned in a blood vessel, the expandable body 12 may then be expanded to press the tube 2 against the against the vessel wall in a stent-like fashion. Thus, the expandable body 12 provides structural support for the tube 2 without occupying the inner lumen of the tube 2. This may provide a luminal surface which more readily encourages growth of a cellular carpet. The inverted proximal end also provides a thickness which may reduce leakage between apparatus and the vessel wall.

It may be appreciated that the expandable body 12 may alternatively be embedded in the tube wall 8 or positioned within the tube 2 so that the microstructures 14 penetrate into and optionally through the wall 8 radially outwardly from the tube 2. Again, the first end 4 may be inverted to cover the microstructures 14. Further, the microstructures 14 may optionally be long enough to penetrate through the inverted first end 4 to then penetrate into the surrounding vessel wall.

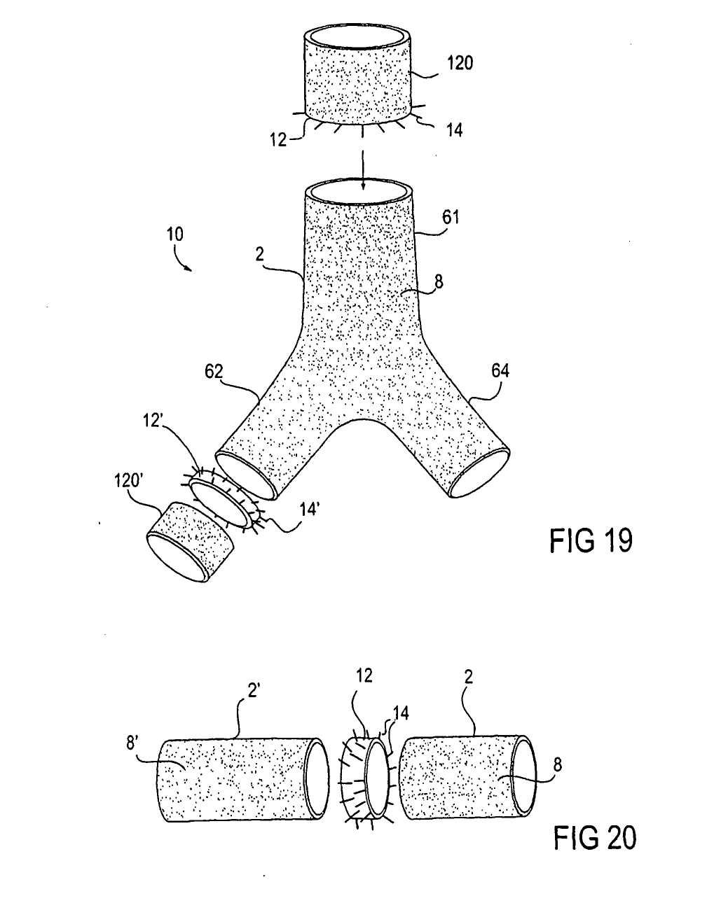

In another example, shown in FIG. 19, the apparatus 10 of the present invention is also shaped to fit within the abdominal aorta, traversing the bifurcation. Again, the tube 2 includes a main shaft 61, a first leg 62, and a second leg 64. In addition, this embodiment includes extension cuffs 120. Extension cuffs 120 are used to lengthen select portions of the apparatus 10 to accommodate various anatomical differences in geometries or procedural changes during surgical operations, to name a few. Extension cuffs 120 are typically comprised of the same or similar material as the tube 2 of the apparatus 10. The extension cuffs 120 are joined with the tube 2 with the use of an expandable body 12 having deployable microstructures 14. The expandable bodies 12 may be attached to the extension cuff 120 either on an external surface of the cuff, an internal surface of the cuff or embedded within the wall of the cuff. FIG. 19 illustrates an extension cuff 120 having an expandable body 12 thereattached wherein the extension cuff 120 is then connectable with the main shaft 61. Alternatively, the bodies 12 maybe separate from the tube 2 and insertable within the tube 2 or the extension cuff 120 so that the microstructures 14 penetrate through the walls of the tube 2 and cuff 120. Thus, FIG. 19 also illustrates an extension cuff 120′ which is separate from and connectable with an expandable body 12′ and first leg 62.

In any case, the microstructures 14 may take any form described above and be deployable to project radially outwardly. The microstructures 14 then penetrate the extension cuff 120, 120′ and/or the tube 2 to attach the cuffs 120 to the tube 2. The microstructures 14 may only partially penetrate or may penetrate through and continue to penetrate through to the surrounding vessel when positioned in a patient. It may be appreciated that although extension cuffs 120, 120′ are illustrated near the ends of each of the main shaft 61 and first leg 62, respectively, cuffs 120, 120′ may be utilized at any or all of the ends of the device 10. Further, as illustrated in FIG. 20, an expandable member 12 may similarly be used to connect two tubes 2,2′. Again, the expandable body 12 may be attached to either of the tubes 2,2′ on an external surface, an internal surface or embedded within the wall of one of the tubes 2,2′. Alternatively, the bodies 12 may be separate from the tubes 2,2′ and insertable within one of the tubes 2,2′ so that the microstructures 14 penetrate through its wall 8,8′. It may be appreciated that either of the tubes 2,2′ may be an extension cuff 120.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

What is claimed is:

1. An apparatus for repair of an aneurysm in a blood vessel of a patient comprising:
a tube having a first end, a second end and a wall extending between the first and second ends, the tube shaped to be disposed at least partially within the aneurysm; and
at least one expandable body attached to the tube wall including at least one microstructure having an attached end attached to the body and a free end in an undeployed position, wherein expansion of the at least one expandable body creates forces which deploy the at least one microstructure from the undeployed position to a deployed position wherein the free end of the at least one microstructure projects radially outwardly from the tube.

2. An apparatus as in claim 1, wherein the at least one expandable body is attached to an exterior surface of the tube wall.

3. An apparatus as in claim 1, wherein the at least one expandable body is embedded within the tube wall.

4. An apparatus as in claim 1, wherein the at least one expandable body is attached to an interior surface of the tube wall.

5. An apparatus as in claim 1, wherein the at least one microstructure comprises a plurality of microstructures positioned to project radially outwardly from the tube near the first end, the second end or near both ends.

6. An apparatus as in claim 1, wherein the at least one microstructure projects radially outwardly from the tube a distance sufficient to penetrate the blood vessel to reduce migration of the apparatus within the blood vessel.

7. An apparatus as in claim 1, wherein the at least one microstructure comprises a plurality wherein the plurality of microstructures are arranged to reduce leakage between the apparatus and the blood vessel.

8. An apparatus as in claim 1, wherein the blood vessel comprises a segment of an aorta having two iliac arteries therewith at an aortic bifurcation, and wherein the tube further comprises an opening between the first end and the second end to align with one of the iliac arteries.

9. An apparatus as in claim 8, wherein the at least one microstructure further comprises a plurality of microstructures positioned to project radially outwardly from the tube around the opening.

10. An apparatus as in claim 1, wherein the blood vessel comprises a segment of an aorta having two iliac arteries therewith at an aortic bifurcation, and wherein the tube is shaped to be disposed within one of the two iliac arteries and to connect with another tube positioned within the segment of the aorta.

11. An apparatus as in claim 10, wherein the plurality of microstructures project radially outwardly from the tube a distance sufficient to penetrate the another tube to attach the tube to the another tube.

12. An apparatus as in claim 11, wherein the distance is insufficient to penetrate through and extend beyond the another tube.

13. An apparatus as in claim 11, wherein the distance is sufficient to additionally penetrate the aorta.

14. An apparatus as in claim 1, further comprising a material carried by the at least one microstructure, wherein the material is delivered to the patient by the at least one microstructure.

15. An apparatus as in claim 14, wherein the material comprises DNA, a drug, VEGF, thrombin, collagen or any combination of these.

16. An apparatus as in claim 14, wherein the material is coated on a surface of the at least one microstructure.

17. An apparatus as in claim 14, wherein the material is held in a lumen within the at least one microstructure.

18. An apparatus as in claim 1, wherein the at least one expandable body has a proximal end, a distal end, and a longitudinal axis therebetween, and wherein the at least one microstructure comprises a plurality of microstructures, each microstructure having first and second supports affixed to associate first and second adjacent portions of the radially expandable body,
expansion of the expandable body within the patient effecting relative movement between the associated first and second portions of the expandable body,
the relative movement deploying the microstructures to the deployed position with the free end projecting radially outwardly from the longitudinal axis.

19. An apparatus as in claim 18, wherein the free end has a pointed shape.

20. An apparatus as in claim 19, wherein the pointed shape includes a single point or a multiple point.

21. An apparatus as in claim 19, wherein the free end has an arrow shape including a pointed tip and at least one undercut that resists withdrawal of the free end from the blood vessel.

22. An apparatus as in claim 18,wherein the relative movement of the associated first and second portions of the expandable body comprises circumferential movement of the first portion relative to the second portion when the expandable body expands radially.

23. An apparatus as in claim 22, wherein the circumferential movement pulls the affixed ends of the first and second supports apart which moves the free end.

24. An apparatus as in claim 18, wherein the first and second supports comprise elongate shafts extending between the free end and the associated first and second adjacent portions of the radially expandable body.

25. An apparatus as in claim 24, wherein the relative movement of the associated first and second portions of the expandable body comprises moving the associated first and second portions apart so that the supports pull the free end in opposite directions causing the free end to project radially outwardly.

26. A system for repair of an aneurysm in a blood vessel in a patient comprising:
a tube having a first end, a second end and a wall extending between the first and second ends, the tube shaped to be disposed at least partially within the aneurysm; and
a first expandable body having a proximal end, a distal end, a longitudinal axis therebetween, and at least one microstructure having an attached end attached to the body and a free end in an undeployed position, expansion of the body creating forces which deploy the at least one microstructure from the undeployed position to a deployed position wherein the free end projects radially outwardly from the longitudinal axis,
the first expandable body sized for positioning within the tube so that expansion of the body penetrates the at least one microstructures through the tube wall.

27. A system as in claim 26, wherein the at least one microstructure projects radially outwardly from the tube a distance sufficient to penetrate the blood vessel.

28. A system as in claim 26, wherein the free end has a pointed shape.

29. A system as in claim 28, wherein the pointed shape includes a single point or a multiple point.

30. A system as in claim 28, wherein the free end has an arrow shape including a pointed tip and at least one undercut that resists withdrawal of the free end from the blood vessel.

31. A system as in claim 26, wherein the first expandable body is configured for positioning within the first end of the tube.

32. A system as in claim 31, further comprising a second expandable body configured for positioning within the second end of the tube,
the second expandable body having a proximal end, a distal end, a longitudinal axis therebetween, and at least one microstructure having an attached end attached to the body and a free end in an undeployed position, expansion of the second expandable body creating forces which deploy the at least one microstructure from the undeployed position to a deployed position wherein the free end projects radially outwardly from the longitudinal axis,
the second expandable body sized for positioning within the tube so that expansion of the body penetrates the at least one microstructures through the tube wall.

33. A system as in claim 32, wherein the blood vessel comprises a segment of an aorta having two iliac arteries therewith at an aortic bifurcation, and wherein the tube is shaped to be disposed within the aortic segment and the tube further comprises an opening between the first end and the second end to align with one of the iliac arteries.

34. A system as in claim 33, further comprising another tube shaped to be disposed within the one of the iliac arteries and to extend through the opening.

35. A system as in claim 34, further comprising a third expandable body configured for positioning within the another tube,
the third expandable body having a proximal end, a distal end, a longitudinal axis therebetween, and at least one microstructure having an attached end attached to the body and a free end in an undeployed position, expansion of the second expandable body creating forces which deploy the at least one microstructure from the undeployed position to a deployed position wherein the free end projects radially outwardly from the longitudinal axis,
the third expandable body sized for positioning within the another tube so that expansion of the body penetrates the at least one microstructures through the another tube wall.

36. A system as in claim 26, wherein further comprising a material carried by the at least one microstructure, wherein the material is delivered to the patient by the at least one microstructure.

37. A system as in claim 36, wherein the material comprises DNA, a drug, VEGF, thrombin, collagen or any combination of these.

38. A system as in claim 36, wherein the material is coated on a surface of the at least one microstructure.

39. A system as in claim 36, wherein the material is held in a lumen within the at least one microstructure.

40. A method of treating an aneurysm in a blood vessel of a patient comprising the steps of:
providing an apparatus comprising a tube having a first end, a second end and a tube wall extending between the first and second ends, and at least one expandable body attached to the tube wall including at least one microstructure having first and second supports and a free end, the supports affixed to associate first and second adjacent portions of the at least one expandable body;
positioning the apparatus within the blood vessel and so that it extends across the aneurysm, wherein the at least one microstructure is in an undeployed position; and
expanding the at least one expandable body effecting relative movement between the associated first and second adjacent portions of the expandable body, the relative movement deploying at least one microstructure from the undeployed position to a deployed position wherein the at least one microstructure projects radially outwardly from the tube.

41. A method as in claim 40, further comprising expanding the at least one expandable body so that the deployed at least one microstructure penetrates a wall of the blood vessel.

42. A method as in claim 41, wherein the deployed at least one microstructure penetrates a wall of the blood vessel so that migration of the apparatus within the blood vessel is reduced.

43. A method as in claim 41, wherein the at least one microstructure comprises a plurality of microstructures in a predetermined arrangement, and wherein the deployed at least one microstructure penetrates a wall of the blood vessel so that the predetermined arrangement reduces leakage between the apparatus and the blood vessel.

44. A method as in claim 40, wherein the at least one expandable body comprises a first expandable body disposed near the first end and a second expandable body disposed near the second end, and wherein positioning the apparatus comprises positioning the first and second expandable bodies so that the aneurysm lies between the first and second expandable bodies.

45. A method as in claim 40, wherein the blood vessel comprises a segment of an aorta having two iliac arteries therewith at an aortic bifurcation and wherein the tube further comprises an opening between the first end and the second end, the method further comprising aligning the opening with one of the iliac arteries.

46. A method as in claim 45, further comprising positioning an iliac graft within the one of the iliac arteries so that a portion of the iliac graft passes through the opening to connect with the apparatus.

47. A method as in claim 45, wherein the iliac graft further comprises at least one expandable body including at least one microstructure having an attached end attached to its body and a free end, further comprising expanding the at least one expandable body of the iliac graft to deploy its at least one microstructure so that its free ends project radially outwardly through the wall of the apparatus to join the iliac graft to the apparatus.

48. A method as in claim 40, wherein the at least one microstructure carries a material and further comprising delivering the material to the patient.

49. A method as in claim 48, wherein the material is coated on a surface of the at least one microstructure and delivering the material comprises diffusion of the material from the surface of the at least one microstructure to the blood vessel.

50. A method as in claim 49, wherein delivering the material comprises diffusion of the material from the surface of the at least one microstructure to the aneurysmal sac.

51. A method as in claim 48, further comprising expanding the body so that the deployed at least one microstructure penetrates a wall of the blood vessel, wherein the material is coated on a surface of the at least one microstructure and delivering the material comprises transferring the material from the surface of the at least one microstructure to the penetrated blood vessel wall.

52. A method as in claim 48, further comprising expanding the body so that the deployed at least one microstructure penetrates a wall of the blood vessel, wherein the material is held in a lumen within the at least one microstructure, and delivering the material comprises injecting the material into the penetrated blood vessel wall.

53. A method as in claim 48, wherein the material comprises DNA, a drug, VEGF, thrombin, collagen or any combination of these.

54. A system for repair of an aneurysm in a blood vessel of a patient comprising:
a tube having a first end, a second end and a wall extending between the first and second ends, the tube shaped to be disposed at least partially within the aneurysm; and
an extension cuff having at least one expandable body attached to the cuff, the expandable body including at least one microstructure having an attached end attached to the body and a free end in an undeployed position, wherein expansion of the at least one expandable body creates forces which deploy the at least one microstructure from the undeployed position to a deployed position wherein the free end of the at least one microstructure projects radially outwardly from the cuff and penetrates the wall of the tube so as to attach the cuff with the tube.

55. An system as in claim 54, wherein the at least one expandable body is attached to an exterior surface of the extension cuff.

56. An system as in claim 54, wherein the at least one expandable body is embedded within a wall of the extension cuff.

57. An system as in claim 54, wherein the at least one expandable body is attached to an interior surface of the extension cuff.

58. An system as in claim 54, wherein the blood vessel comprises a segment of an aorta having two iliac arteries therewith at an aortic bifurcation and the tube is shaped to have a main shaft, a first leg and a second leg.

59. An system as in claim 54, wherein penetration is insufficient to penetrate through and extend beyond the wall of the tube.

60. A system for repair of an aneurysm in a blood vessel of a patient comprising:
a tube having a first end, a second end and a tube wall extending between the first and second ends, the tube shaped to be disposed at least partially within the aneurysm;
a first expandable body having a proximal end, a distal end, a longitudinal axis therebetween, and at least one microstructure having an attached end attached to the body and a free end in an undeployed position, expansion of the body creating forces which deploy the at least one microstructure from the undeployed position to a deployed position wherein the free end projects radially outwardly from the longitudinal axis; and
an extension cuff having a cuff wall shaped to be disposed within the blood vessel;
the first expandable body sized for positioning within the tube and the cuff so that expansion of the body penetrates the at least one microstructures through the tube wall and cuff wall as to attach the cuff with the tube.

61. A method of treating an aneurysm in a blood vessel of a patient comprising the steps of:
providing a tube shaped to be disposed within an aneurysm;
positioning the tube within the blood vessel and so that it extends across the aneurysm;
providing an extension cuff having a cuff wall and at least one expandable body attached to the cuff wall, the expandable body including at least one microstructure having an attached end attached to the body and a free end in an undeployed position;
positioning the cuff within the blood vessel and so that it mates with the tube, wherein the at least one microstructure is in an undeployed position; and
deploying the at least one microstructure to a deployed position wherein the at least one microstructure projects radially outwardly from the cuff and penetrates the wall of the tube so as to attach the cuff with the tube.
